(19) United States
(12) Patent Application Publication
(10) Pub. No.: US 2026/0240978 A1
(43) Pub. Date: Aug. 20, 2026

(54) SYSTEMS AND METHODS FOR PREDICTING VACCINE SENTIMENT

(71) Applicant: STChealth, LLC, Phoenix, AZ (US)

(72) Inventors: Nathanael Meckes, Phoenix, AZ (US); Sawyer Koops, Phoenix, AZ (US); Kyle Freese, Phoenix, AZ (US); Samuel Mcgee, Phoenix, AZ (US)

(73) Assignee: STChealth, LLC, Phoenix, AZ (US)

(21) Appl. No.: 19/541,652

(22) Filed: Feb. 17, 2026

Related U.S. Application Data
(60) Provisional application No. 63/760,545, filed on Feb. 19, 2025.

(51) Int. Cl. A61K 39/12; G06N 5/01; G06N 20/20

(52) U.S. Cl. CPC A61K 39/12; G06N 5/01; G06N 20/20

(57) ABSTRACT

Systems and methods for determining vaccine sentiment of a patient are described. The systems and methods can include creating one or more training datasets from patient data, training one or more predictive algorithms to generate a vaccine sentiment score, using one or more predictive algorithms to generate a vaccine sentiment score, and generating one or more vaccine related communications based on a vaccine sentiment score.

### CROSS-REFERENCE TO RELATED APPLICATIONS

This application is a non-provisional of and claims priority to U.S. Provisional Application No. 63/760,545, filed Feb. 19, 2025 and entitled “SYSTEMS AND METHODS FOR PREDICTING VACCINE SENTIMENT,” which is herein incorporated by this reference in its entirety.

### TECHNICAL FIELD

This disclosure relates generally to predictive algorithms and more particularly to predictive algorithms for medical and/or public health applications

### BACKGROUND

Measuring vaccine sentiment in a population is a well know problem within the medical arts that has been present since the introduction of the first smallpox vaccine. While vaccine sentiment (e.g., a desire to receive and/or not receive a vaccine) is well documented in medical literature, data about vaccine sentiment can come from survey data and/or county-level vaccination data. While these types of data on their own can aid in predicting the number of people likely to get vaccinated, it cannot accurately predict vaccine sentiment at the vaccine and/or patient level. This, in turn has ramifications for public health departments, vaccine manufacturers, and even school systems.

Further, in the field of predictive algorithms, training data is used for developing accurate and reliable models. High-quality training data allows models and/or algorithms to learn patterns, make predictions, and determine a patient's vaccine sentiment. However, generating effective training data from raw and/or partially processed data presents significant challenges. For example, inherent complexities and variability within the raw and/or partially processed data can lead to errors and poor predictions of sentiment. Raw and/or partially processed data (e.g., logs, user inputs, and/or public data sources) can be unstructured, noisy, and/or can lack consistent formatting. This raw and/or partially processed data may contain incomplete records, outliers, or irrelevant information that can negatively impact model performance if incorporated into a training dataset. For instance, raw text data may include typos, non-standard abbreviations, medical jargon, or irrelevant words, while partially processed data can contain extraneous objects or inconsistent notations. Processing this data to be consistent and relevant can involve extensive cleaning, labeling, and/or formatting to ensure it meets the needs of the intended machine learning application.

Therefore, in view of the above, there is a need for a new system and method for a predictive algorithm that can determine a patient's vaccine sentiment.

### BRIEF DESCRIPTION OF THE DRAWINGS

To facilitate further description of the embodiments, the following drawings are provided in which:

FIG. 1 illustrates a flowchart for a method, according to an embodiment;

FIG. 2 illustrates a representative block diagram of a system, according to an embodiment; and

FIG. 3 illustrates a representative block diagram of a system, according to an embodiment.

### DESCRIPTION OF EXAMPLES OF EMBODIMENTS

A number of embodiments can include a system. The system can include one or more processors and one or more non-transitory computer-readable storage devices storing computing instructions. The computing instructions can be configured to run on the one or more processors and cause the one or more processors to perform: generating one or more training datasets; training a predictive algorithm on the one or more training datasets; receiving new patient data of a patient; determining, using the trained predictive algorithm and the new patient data, a vaccine sentiment score; applying a label to the patient using the vaccine sentiment score; and coordinating displaying a vaccine communication using the label.

Various embodiments include a method. The method can be implemented via execution of computing instructions configured to run at one or more processors and configured to be stored at non-transitory computer-readable media The method can comprise generating one or more training datasets; training a predictive algorithm on the one or more training datasets; receiving new patient data of a patient; determining, using the trained predictive algorithm and the new patient data, a vaccine sentiment score; applying a label to the patient using the vaccine sentiment score; and coordinating displaying a vaccine communication using the label.

Many embodiments can include an article of manufacture. The article of manufacture can comprise a non-transitory, tangible computer readable storage medium having instructions stored thereon that, in response to execution by a computer based system, cause the computer based system to perform operations comprising generating one or more training datasets; training a predictive algorithm on the one or more training datasets; receiving new patient data of a patient; determining, using the trained predictive algorithm and the new patient data, a vaccine sentiment score; applying a label to the patient using the vaccine sentiment score; and coordinating displaying a vaccine communication using the label.

In many embodiments, the techniques described herein can provide a practical application and several technological improvements. In some embodiments, the techniques described herein can provide for improved identification of vaccine sentiment in patients and/or geographical areas. These techniques can provide a significant improvement over conventional approaches of identifying vaccine sentiment, such as interviewing a patient. In many embodiments, the techniques described herein can beneficially make determinations based on dynamic information that describes current conditions and/or conditions that have occurred during the same day of a patient's vaccine appointment. In this way, the techniques can avoid problems with stale and/or outdated predictive algorithms or training datasets by continually updating.

In many embodiments, the techniques described herein can be used continuously at a scale that cannot be reasonably performed using manual techniques or the human mind. For example, training a predictive algorithm involves datasets too large to for the training sequence to be practically performed in the mind of a human.

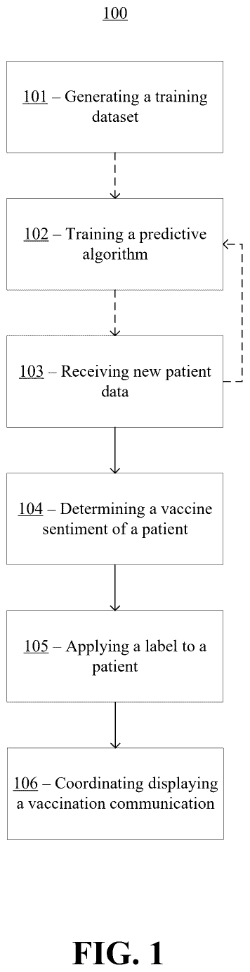

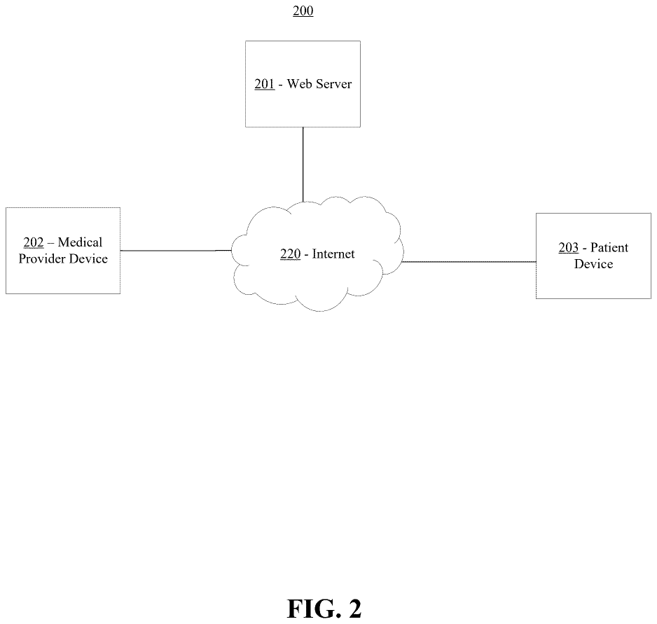

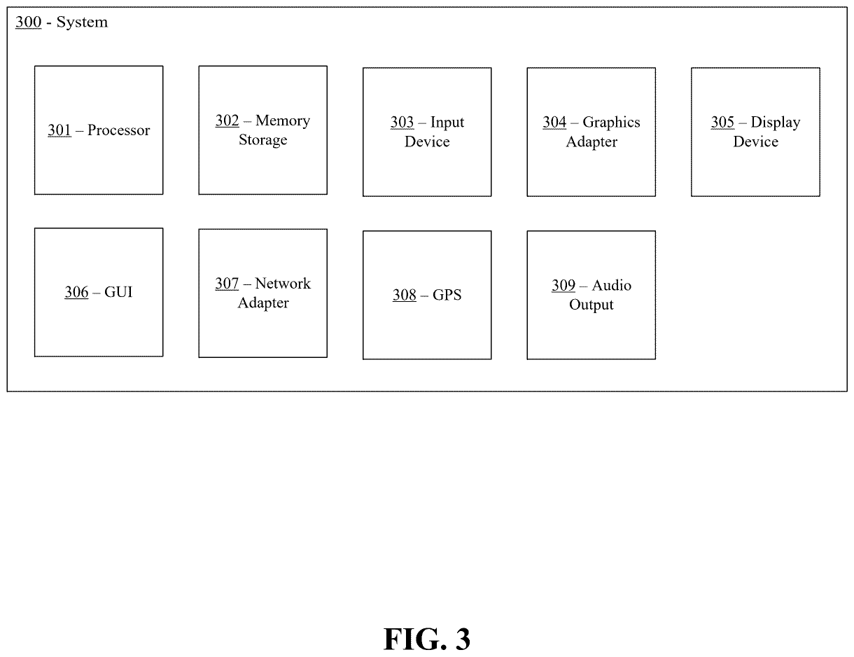

Turning ahead in the drawings, FIG. 1 illustrates a flow chart for a method 100, according to an embodiment. Method 100 is merely exemplary and is not limited to the embodiments presented herein. Method 100 can be employed in many different embodiments or examples not specifically depicted or described herein. In some embodiments, the activities of method 100 can be performed in the order presented. In other embodiments, the activities of method 100 can be performed in any suitable order. In still other embodiments, one or more of the activities of method 100 can be combined or skipped. In many embodiments, system 300 (FIG. 3) can be suitable to perform method 100 and/or one or more of the activities of method 100. In these or other embodiments, one or more of the activities of method 100 can be implemented as one or more computer instructions configured to run at one or more processing modules and configured to be stored at one or more non-transitory memory storage modules. Such non-transitory memory storage modules can be part of a computer system such as web server 201, medical provider device 202, and/or patient device 203 (FIG. 2). The processing module(s) can be similar or identical to the processing module(s) described above with respect to computer system 300 (FIG. 3).

In some embodiments, method 100 can optionally comprise a step 101 of generating a training dataset. In some embodiments (e.g., when a training dataset is already present or training is not desired), step 101 can be skipped. A training dataset can comprise a set of examples, data, and/or observations about one or more patients used to train a predictive algorithm. From a computational perspective, a training data set can comprise data that is reorganized, reformatted, and/or processed such that it can be ingested by a predictive algorithm. In many embodiments, one or more of raw, processed, and/or partially processed data can be used as a training data set. Raw, processed, and/or partially processed data can be received from a number of sources and then converted into and/or used as training data (when sufficiently processed) in a training data set. For example, raw, processed, and/or partially processed data can be received from a governmental (e.g., national level data, state level data, county or parish level data, city level data, etc.) source. In a more specific example, raw, processed, and/or partially processed data can be received from one or more health departments (e.g., Centers for Disease Control (CDC), Department of Health and Human Services (DHHS), Arizona Health Care Cost Containment System (AHCCCS), etc.). As a further example, raw, processed, and/or partially processed data can be purchased from a data broker. In some embodiments, a completed training data set can be received from an external source. For example, a training data set can be purchased from a data broker in a labeled or unlabeled form. In embodiments where unlabeled data is received in step 101, a label can be applied to the data before it is incorporated into a training dataset. For example, one or more annotators can manually label patient data received in step 101 with either a vaccine sentiment score and/or a label generated from a vaccine sentiment score. As another example, patient data received in step 101 can be run through a labeling algorithm that labels the data with a vaccine sentiment score and/or specific label.

Raw, processed, and/or partially processed data can contain a number of data points describing a patient's demographic data (age, race, language, ethnicity, country/area of origin, etc.), a health history, and/or location associated with a patient. For example, health history data can comprise vaccination data comprising a list of vaccination events, vaccine type, vaccine administration location (e.g., country, state, region, county, city, parish, zip code, etc.), and/or vaccination date. Location data can comprise all or a portion of an address for the patient (e.g., home, business, school, etc.) and/or GPS coordinates. Raw, processed, and/or partially processed data can be stored as structured tables (as described in further detail below) in a database for predictive algorithm ingestion, but can also be received as flat data (e.g., a delimited .txt or .csv file). In some embodiments, flat data can be formatted according to all or a part of a specification shown in Table 1.

TABLE 1

| Variable Name | Description | Format |
|---|---|---|
| UNIQUE_ID | Unique ID for a patient | String |
| PAT_FIRST_NAME | Patient's first name | String |
| PAT_LAST_NAME | Patient's last name | String |
| PAT_BIRTH_DATE | Patient's date of birth | Date/Time (e.g., |
| | | yyyy-mm-dd) |
| PAT_GENDER | Patient's gender | String |
| PAT_RACE | Patient's race (e.g., | String |
| | as coded by CDC | |
| | guidelines) | |
| PAT_ETHNICITY | Patient's ethnicity (e.g., | String |
| | as coded by CDC | |
| | guidelines) | |
| ADDRESS_STREET1 | Patient's street address | String |
| ADDRESS_CITY | Patient's city of residence | String |
| ADDRESS_ZIP | Patient's zip code | String |
| VACCINE_CVX | CDC CVX number for a | String |
| | received vaccine | |
| VACC_DATE | Date a vaccine was | Date/Time (e.g., |
| | administered | yyyy-mm-dd) |

Raw, processed, and/or partially processed data can contain one or more features. A feature can comprise an attribute of a patient that contributes to their vaccine sentiment. For example, a feature can comprise a patient's vaccination history, a patient's demographic data, a patient's location (e.g., address and/or GPS) data, a patient's education data (e.g., for the patient's county, state, city, etc.), and/or census data (e.g., for the patient's county, state, city, etc.). Raw, processed, and/or partially processed data can be refined into a training data set in a number of ways. When received, raw, processed, and/or partially processed data can be received in a tabular format (e.g., as one or more tables, databases, and/or spreadsheets). Rows in a tabular format can be associated with one or more patients, vaccine events (e.g., a vaccination clinic, in office vaccination, etc.), dates, and/or locations. Columns in a tabular format can be associated with one or more vaccine names, vaccine types, and/or location data (e.g., address, county, etc.).

Due to a variability in how some location data is entered, raw, processed, and/or partially processed location data can be processed and/or cleaned to generate all or a portion of a training data set. Raw location data can include one or more extraneous identifiers ill-suited for ingestion by a predictive algorithm. In these embodiments, the extraneous identifiers can be removed to produce ingestible identifiers. For example, many sets of location data can contain the term “county” and/or “parish” when describing their location. These terms can be removed from the location data to produce a list of county (or parish) names. Further, some location data can trigger a null variable in a county and/or parish data frame. In these embodiments, an entry for county and/or parish can be set to a predetermined county (or parish) name or a county (or parish) name can be identified using a zip code data field. For example, zip codes 46213 and/or 46288 can produce a null variable for a county and can be set to “Marion.” In this way, patients who are associated with non-residential zip codes can be associated with a county of residence. White space can also be removed from location data and capital letters can be demoted to lowercase to increase a uniformity in the location data.

In many embodiments, raw, processed, and/or partially processed vaccination history data can be processed (or further processed) to generate all or a portion of a training data set. Vaccination history data can be processed to generate one or more new columns in a tabular format. For example, a time since a last vaccination for a patient can be determined using a vaccine history for a patient. Each portion of a date for a patient's last vaccine can be broken into a new column (e.g., day, month, and/or year). A count of a number of days since a previous vaccination can then be calculated by subtracting a day column from a variable for a current day that is initialized in a configuration file. After being subtracted, a resulting number of days since a last vaccination can be converted into a number of months since a last vaccination (e.g., by dividing by 30 or 31).

Each patient row can a comprise columns for one or more CDC defined vaccine families. A 1 (or some other suitable notation) can be present when a patient has been administered with a vaccine and a 0 (or some other suitable notation) can be present if there are no records found to indicate that the patient has received that vaccine. Some seasonal vaccines (e.g., flu and/or COVID vaccines) can have a column for each disease season (e.g., 2019 versus 2020 flu season). These seasonal columns can be used to determine a time period between administration of seasonal vaccines. This time period can be initialized in a new column for seasonal vaccines by subtracting a day column from a variable for a current day that is initialized in a configuration file. For non-seasonal vaccines, a 999 (or some other suitable notation) can be entered into a seasonal time period column. During training, non-seasonal vaccines can be identified using the notation so that they can be dropped, ignored, and/or weighted such that they do not influence downstream predictions. A new column can also be created for vaccinations where more than one dose is administered. For example, a COVID-19 series of vaccinations can be counted in three separate columns. In some embodiments, vaccination history for patients under 18 years of age can be dropped, ignored, and/or weighted in a training dataset such that they do not influence downstream predictions. In many embodiments, a vaccine history portion of a training data set can comprise a row for each vaccine taken by a patient. For example, a vaccine history portion of a training data set in tabular form can comprise a number of rows corresponding to a total number of vaccines given and can have a number of columns corresponding to relevant patient information. In this way, training data can be used to generate accurate predictions on an individual vaccine level.

In many embodiments, raw, processed, and/or partially processed feature data can be updated. In this way, training data can become more refined and better suited for creating accurate predictions of vaccine sentiment. For example, redundant data entries can be removed either from raw, processed, and/or partially processed training data and/or redundant data frames can be removed from the raw, processed, and/or partially processed training data. In a more specific embodiment, columns containing a birthdate can be removed because it is duplicative with a column containing a patient's age. As another example, float type data frames can be converted to integer type data frames, thereby increasing storage efficiency. As a further example, overlapping racial and ethnic data can be merged into one column, data frame, and/or data entry. Due to the simpler data formats (e.g., a single number for age vs a multi-part data frame for birthday), removing redundancies and/or complexities can increase a speed of a training process and/or increase storage efficiency for a training dataset.

In some embodiments, method 100 can optionally comprise a step 102 of training a predictive algorithm. In various embodiments (e.g., when training is not desired), step 102 can be skipped. A predictive algorithm can be trained on one or more training datasets (e.g., those received and/or generated in step 101). In many embodiments, training a predictive algorithm can comprise estimating internal parameters of a model configured to generate a vaccine sentiment score. In this way, a predictive algorithm can be configured to determine when a patient and/or a plurality of patients are likely to be receptive to a vaccine or resistant to a vaccine. In the same or different embodiments, a pre-trained predictive algorithm can be used, and the pre-trained algorithm can be re-trained on one or more training datasets. In some embodiments, the predictive algorithm can consider both historical and dynamic input from state agencies when electronic medical records are received iteratively and/or as a data stream. In this way, a predictive algorithm can be trained iteratively as new data is added to a training data set. In many embodiments, a predictive algorithm can be iteratively trained in real time as data is added to a training data set. In various embodiments, a predictive algorithm can be trained, at least in part, on a single patient's health and/or demographic data or the single patient's health and/or demographic data can be weighted in a training data set. In this way, a predictive algorithm tailored to a single user can be generated. In the same or different embodiments, a predictive algorithm tailored to a single user can be used as a pre-trained algorithm for a similar user. In several embodiments, due to a large amount of data needed to create and maintain a training data set, a predictive model can use extensive data inputs to generate a vaccine sentiment score. Due to these extensive data inputs, in many embodiments, creating, training, and/or using a predictive algorithm configured to generate a vaccine sentiment score cannot practically be performed in a mind of a human being.

In many embodiments, a predictive algorithm can comprise a machine learning algorithm. In various embodiments, a machine learning algorithm can comprise a decision tree. A decision tree can comprise one or more analytic nodes connected by one or more branches. A number of different types of nodes can be present in a decision tree. For example, a node can be a decision node or a leaf node. In some embodiments, a decision node in a decision tree algorithm can classify data points into two or more classifications. For example, a decision node can classify a transaction based on a value of the transaction. In these or other embodiments, a leaf node can comprise a node where data points are of the same or similar classification. In this way, data points can be classified by being fed into a first decision node of a decision tree (known as a root node) and then passed through the branches to other decision nodes and leaf nodes. In various embodiments, classification of a datapoint can end when it reaches a leaf node. In some embodiments, a decision tree model can comprise a random forest model. A random forest model can comprise an algorithm in which multiple decision trees are used in parallel to classify and/or label a data point. In various embodiments, a decision tree algorithm can comprise a gradient boosted algorithm. Gradient boosting is an ensemble learning method that builds consecutive small trees (some as small as one node), where each consecutive tree is configured to correct an error from a previous tree. In some embodiments, an optimization algorithm can be used in combination with a boosted decision tree, thereby creating a gradient boosted algorithm. In various embodiments, an optimization algorithm can be configured to minimize a cost function (e.g., an error or a loss). In this way, a decision tree algorithm can be made more accurate than other predictive algorithms. A number of different cost functions can be used in step 102. For example, an accuracy function can be used. An accuracy function can use a number of true positives, a number of true negatives, a number of false positives, and a number of false negatives to determine a cost. For example, an accuracy function can comprise:

$$Cost = \frac{\# of true positives + \# of true negatives}{\begin{matrix}\# of true positives + \# of true negatives + \\ \# of false positives + \# of false negatives\end{matrix}}$$

In many embodiments, method 100 can comprise a step 103 of receiving new patient data. New patient data can be received from a number of sources. For example, new patient data can be received from a data broker, a governmental body, or some other third party. As another example, new patient data can be received directly from a patient and/or a patient's healthcare provider. A patient can enter their data into a website, use an electronic medical record, orally provide their data, and/or supply their data on a traditional paper medical record. New patient data can be received from one or more of web server 201 (FIG. 2), medical provider device 202 (FIG. 2), and/or patient device 203 (FIG. 2). New patient data can comprise additional training data for inclusion in a training dataset and/or information for a single patient or a plurality of patients for analysis in a predictive algorithm. In some embodiments, new patient data can comprise one or more of raw, partially processed, and/or processed data. New patient data can be process and/or refined as described above. Features and/or labels can be extracted from the new patient data as described above. In some embodiments, new patient data can comprise one or more event locations. For example, new patient data can be appended to one or more pieces of location data at a state, county (or parish), city, and/or zip code level. When additional training data is received, a predictive algorithm can be made more accurate by re-training a predictive algorithm on the new patient data. For example, when new patient data comprises data about a new vaccine, a predictive model can be updated to predict a vaccine sentiment score for the new vaccine. When analysis of new patient data is desirable, the new patient data can be inputted into a predictive algorithm for determination of a vaccine sentiment score and/or a label.

In many embodiments, method 100 can comprise step 104 of determining a vaccine sentiment score. In some embodiments, step 104 can be performed concurrently with one or more of activities 101-103. In various embodiments, step 104 can be performed in response to completing one or more of activities 101-103. New patient data can be passed into a predictive algorithm (e.g., a gradient boosted decision tree) as an input and a vaccine sentiment score can be outputted. For example, if an output of a predictive algorithm is a measurement of loss, then a lower output can indicate lower vaccine sentiment. As another example, if an output of a predictive algorithm is a likelihood of vaccine hesitancy, then a higher output can indicate more vaccine hesitancy. While higher sentiment scores are described herein as indicating a higher likelihood that a patient will accept a vaccine, it should be understood that the opposite can also be true. For example, a higher vaccine sentiment can, in some embodiments, indicate a lower likelihood of a patient accepting a vaccine. In many embodiments, newly received patient data can be converted into a compliant data format before inputted into a predictive algorithm. For example, one or more portions of step 101 can be performed before new patient data is inputted into a predictive algorithm.

When a predictive algorithm comprises a decision tree, newly received patient data can be inputted into the decision tree and be analyzed by one or more analytic nodes. As the newly received patient data can be processed and/or passed down through the decision tree, signals indicative of a vaccine sentiment score can be discovered and/or refined. When the newly received patient data arrives at a leaf node, a classification in the leaf node can be read as at least a portion of a vaccine sentiment signal. Results, classifications, and/or labels generated by leaf nodes can be aggregated to produce an overall vaccine sentiment score. In many embodiments, each different vaccination can have its sentiment score predicted by its own predictive algorithm, and when multiple vaccines are being offered (e.g., at a vaccine event), a system can run each vaccine's predictive algorithm in parallel. In some embodiments, only one instance of a predictive algorithm is used to determine a sentiment score for one or more available vaccines.

A predictive algorithm can be accessed in a number of different ways. For example, a predictive algorithm can be embedded into a function. An algorithm can be embedded into a function using a scripting language (e.g., Python). This function can comprise an invokable function comprising patient data inputs for one or more entities (e.g., servers, storage modules, etc.) at a time. This, in turn, can allow the algorithm to be called locally from other code or functions stored on a user device, thereby avoiding costs and delays associated with remote and/or cloud computation of vaccine sentiment. Further, locally calling a predictive algorithm can lead to a more accurate prediction that is specific to the individual patient data being processed. A function comprising a predictive algorithm can also be wrapped in a web service endpoint (e.g., a vaccination website). This, in turn, can enable a predictive algorithm to be invoked remotely by users without access to the function locally, thereby increasing security around patient data contained in a training dataset.

In many embodiments, inputs and outputs of a predictive algorithm can remain at a single entity level to preserve patient privacy. In these embodiments, inputs can be accepted for a single patient's first and/or last name, with a single numeric representation of either or both names returned. In many embodiments, an application programming interface (API) can be used to access a predictive algorithm. Using an API to call a predictive algorithm can present a number of advantages. First, an API can allow a predictive algorithm to be invoked over a network connection while still being secured with an authentication of a web service's choice. Second, an API can allow a hosting service for the predictive algorithm to impose any required input formatting controls and error handling that are desired. Finally, an API presents an industry standard front end to a predictive algorithm, which can be used/invoked from other processes and systems.

In many embodiments, method 100 can comprise a step 105 of applying a label to a patient. A label can comprise an output of a predictive algorithm for a specific feature or combination of features and/or can be determined by a patient's vaccine sentiment score. For example, vaccine sentiment scores that fall in predetermined ranges and/or above (or below) a predetermined value can be assigned a specific label. As another example, aggregated features (e.g., countrywide features, countywide features, statewide features, citywide features, etc.) can have a label comprising a percentage of patients in the aggregated feature with a specific label. In many embodiments, a label can be applied to a patient by a predictive model. For example, when a final decision, prediction, or output is generated by a leaf node, the most likely classification (e.g., most likely vaccine sentiment label) can be applied by the patient. As another example, a label can be manually added by a person (e.g., a statistician or healthcare worker) using a vaccine sentiment score. A rules based algorithm can also be invoked to assess a vaccine sentiment score and then add a label according to one or more labeling rules.

In many embodiments, method 100 can comprise a step 106 of coordinating displaying a vaccine communication. In some embodiments, step 106 can be performed in response to completing step 104. In various embodiments, a vaccination communication can comprise a likelihood that a patient will accept a vaccine. A likelihood that a patient will accept a vaccine can be communicated in a number of different ways. For example, a likelihood that a patient will accept a vaccine can be displayed on a vaccine website and/or a vaccine application. As another example, a likelihood that a patient will accept a vaccine can be delivered as an email or some other type of text and/or image based communication format. A likelihood that a patient will accept a vaccine can also take a number of form factors. For example, a likelihood that a patient will accept a vaccine can be displayed as a raw vaccine sentiment score, as predefined buckets (e.g., low vaccine sentiment, moderately low vaccine sentiment, neutral vaccine sentiment, moderately high vaccine sentiment, or very high vaccine sentiment), and/or as a binary output of high sentiment or low sentiment. A vaccine communication can indicate whether a patient is likely to accept a vaccine on a specific day and/or during a specific season. For example, a person who has a low overall vaccine sentiment may have increased vaccine sentiment for a flu vaccine during flu season or during a local spike in flu cases.

A vaccine communication can comprise a list of vaccinations that are due at a given appointment in addition to a likelihood of each vaccination being received during the appointment. In some embodiments, a vaccine communication can comprise one or more vaccine related discussion topics for a medical provider to discuss with their patient. This, in turn, can allow a medical professional to steer a conversation with the patient towards accepting vaccines that are most beneficial to a patient's health. In many embodiments, a vaccine communication can comprise data on a geographic area (e.g., a county, city, or state population) to determine patients likely to receive a vaccination in a given year. In these embodiments, individuals with a vaccine sentiment score above or below a predetermined value (or patients sorted into various labels indicating low vaccine sentiment) in a geographic area can be included in the list. In this way, population areas that may be under covered by vaccinations can be identified. A vaccine communication can then display instructions and/or locations for allocation of vaccine educational resources and/or mobile vaccine clinics. A vaccination communication can also comprise a predictive communication identifying which areas and/or institutions (e.g., schools and/or school districts) will be short of reaching vaccination goals. In this way, medical providers can target care and/or education at specific areas and/or institutions to increase vaccinations to meet the vaccination goal.

Turning ahead in the drawings, FIG. 2 illustrates a block diagram of a system 200 that can be employed for predicting vaccine sentiment, as described in greater detail below. System 200 is merely exemplary and embodiments of the system are not limited to the embodiments presented herein. System 200 can be employed in many different embodiments or examples not specifically depicted or described herein. In some embodiments, certain elements or modules of system 200 can perform various procedures, processes, and/or activities. In these or other embodiments, the procedures, processes, and/or activities can be performed by other suitable elements or modules of system 200. Generally speaking, system 200 can be implemented with hardware and/or software. Part or all of the hardware and/or software implemented in system 200 can be conventional or part or all of the hardware and/or software can be customized (e.g., optimized) for implementing part or all of the functionality of system 200 described herein.

System 200 can include a web server 201 and/or a medical provider device 202. Web server 201 and medical provider device 202 can each be a computer system, such as computer system 300 (FIG. 3), as described below, and can each be a single computer, a single server, a cluster or collection of computers or servers, or a cloud of computers or servers. A single computer system can also host each of two or more of web server 201 and medical provider device 202.

System 200 also can optionally patient device 203. Patient device 203 can comprise any of the elements described in relation to computer system 300 (FIG. 3). For example, user patient device 203 can be a mobile device. A mobile electronic device can refer to a portable electronic device (e.g., an electronic device easily conveyable by hand by a person of average size) with the capability to present audio and/or visual data (e.g., text, images, videos, music, etc.). For example, a mobile electronic device can comprise at least one of a digital media player, a cellular telephone (e.g., a smartphone), a personal digital assistant, a handheld digital computer device (e.g., a tablet personal computer device), a laptop computer device (e.g., a notebook computer device, a netbook computer device), a wearable user computer device, or another portable computer device with the capability to present audio and/or visual data (e.g., images, videos, music, etc.). Thus, in many examples, a mobile electronic device can comprise a volume and/or weight sufficiently small as to permit the mobile electronic device to be easily conveyable by hand. For examples, in some embodiments, a mobile electronic device can occupy a volume of less than or equal to approximately 1790 cubic centimeters, 2434 cubic centimeters, 2876 cubic centimeters, 4056 cubic centimeters, and/or 5752 cubic centimeters. Further, in these embodiments, a mobile electronic device can weigh less than or equal to 15.6 Newtons, 17.8 Newtons, 22.3 Newtons, 31.2 Newtons, and/or 44.5 Newtons. In various embodiments, patient device 203 can comprise a display that is smaller than display device 305 (FIG. 3), thereby facilitating mobility.

Exemplary mobile electronic devices can comprise (i) an iPod®, iPhone®, iTouch®, iPad®, MacBook® or similar product by Apple Inc. of Cupertino, California, United States of America, (ii) a Blackberry® or similar product by Research in Motion (RIM) of Waterloo, Ontario, Canada, (iii) a Lumia® or similar product by the Nokia Corporation of Keilaniemi, Espoo, Finland, and/or (iv) a Galaxy™ or similar product by the Samsung Group of Samsung Town, Seoul, South Korea. Further, in the same or different embodiments, a mobile electronic device can comprise an electronic device configured to implement one or more of (i) the iPhone® operating system by Apple Inc. of Cupertino, California, United States of America, (ii) the Blackberry® operating system by Research In Motion (RIM) of Waterloo, Ontario, Canada, (iii) the Palm® operating system by Palm, Inc. of Sunnyvale, California, United States, (iv) the Android™ operating system developed by the Open Handset Alliance, (v) the Windows Mobile™ operating system by Microsoft Corp. of Redmond, Washington, United States of America, or (vi) the Symbian™ operating system by Nokia Corp. of Keilaniemi, Espoo, Finland.

Further still, the term “wearable user computer device” as used herein can refer to an electronic device with the capability to present audio and/or visual data (e.g., text, images, videos, music, etc.) that is configured to be worn by a user and/or mountable (e.g., fixed) on the user of the wearable user computer device (e.g., sometimes under or over clothing; and/or sometimes integrated with and/or as clothing and/or another accessory, such as, for example, a hat, eyeglasses, a wrist watch, shoes, etc.). A wearable user computer device can comprise a mobile electronic device, and vice versa. However, a wearable user computer device does not necessarily comprise a mobile electronic device, and vice versa.

In specific examples, a wearable user computer device can comprise a head mountable wearable user computer device (e.g., one or more head mountable displays, one or more eyeglasses, one or more contact lenses, one or more retinal displays, etc.) or a limb mountable wearable user computer device (e.g., a smart watch, smart ring, etc.). In these examples, a head mountable wearable user computer device can be mountable in close proximity to one or both eyes of a user of the head mountable wearable user computer device and/or vectored in alignment with a field of view of the user.

In more specific examples, a head mountable wearable user computer device can comprise (i) Google Glass™ product or a similar product by Google Inc. of Menlo Park, California, United States of America; (ii) the Eye Tap™ product, the Laser Eye Tap™ product, or a similar product by ePI Lab of Toronto, Ontario, Canada, and/or (iii) the Raptyr™ product, the STAR 1200™ product, the Vuzix Smart Glasses M100™ product, or a similar product by Vuzix Corporation of Rochester, New York, United States of America. In other specific examples, a head mountable wearable user computer device can comprise the Virtual Retinal Display™ product, or similar product by the University of Washington of Seattle, Washington, United States of America. Meanwhile, in further specific examples, a limb mountable wearable user computer device can comprise the iWatch™ product, or similar product by Apple Inc. of Cupertino, California, United States of America, the Galaxy Gear or similar product of Samsung Group of Samsung Town, Seoul, South Korea, the Moto 360 product or similar product of Motorola of Schaumburg, Illinois, United States of America, and/or the Zip™ product, One™ product, Flex™ product, Charge™ product, Surge™ product, or similar product by Fitbit Inc. of San Francisco, California, United States of America.

Web server 201, medical provider device 202, and/or patient device 203 can each comprise one or more input devices (e.g., one or more keyboards, one or more keypads, one or more pointing devices such as a computer mouse or computer mice, one or more touchscreen displays, a microphone, etc.), and/or can each comprise one or more display devices (e.g., one or more monitors, one or more touch screen displays, projectors, etc.). In these or other embodiments, one or more of the input device(s) can be similar or identical to input device 303 (FIG. 3). Further, one or more of the display device(s) can be similar or identical to display device 305 (FIG. 3) and/or screen 108 (FIG. 1). The input device(s) and the display device(s) can be coupled to the processing module(s) and/or the memory storage module(s) of web server 201, medical provider device 202, and/or patient device 203 in a wired manner and/or a wireless manner, and the coupling can be direct and/or indirect, as well as locally and/or remotely. As an example of an indirect manner (which may or may not also be a remote manner), a keyboard-video-mouse (KVM) switch can be used to couple the input device(s) and the display device(s) to the processing module(s) and/or the memory storage module(s). In some embodiments, the KVM switch also can be part of web server 201, medical provider device 202, and/or patient device 203. In a similar manner, the processing module(s) and the memory storage module(s) can be local and/or remote to each other.

Generally speaking, web server 201 can host one or more websites. For example, web server 201 can host a vaccination website configure to coordinate vaccination schedules and store vaccination records. Web server 201, medical provider device 202, and/or patient device 203 can communicate or interface (e.g., interact) with one another through internet 330. Internet 330 can be an intranet that is not open to the public, a mesh network of individual systems, and/or a distributed system. Accordingly, in many embodiments, web server 201 (and/or the software used by such systems) can refer to a back end of system 200 operated by an operator and/or administrator of system 200, and medical provider device 202 and/or patient device 203 (and/or the software used by such systems) can refer to a front end of system 200 used by one or more medical providers and/or patients, respectively. An operator and/or administrator of system 200 can manage system 200, the processing module(s) of system 200, and/or the memory storage module(s) of system 200 using the input device(s) and/or display device(s) of system 200. Medical providers can also be referred to as customers, in which case, medical provider device 202 can be referred to as a customer device.

Web server 201, medical provider device 202, and/or patient device 203 can also be configured to communicate with one or more databases. The one or more databases can comprise a vaccination database that contains patient vaccine data and/or training datasets used to train a predictive algorithm. Data can be deleted from a database when it becomes older than a maximum age, which can be set by an administrator of system 200. Data collected in real-time can be streamed to a database for storage, thereby increasing a storage speed of a database.

The one or more databases can be stored on one or more memory storage modules (e.g., non-transitory memory storage module(s)), which can be similar or identical to the one or more memory storage module(s) (e.g., non-transitory memory storage module(s)) described above with respect to computer system 300 (FIG. 3). Further, the one or more databases can each be stored on a single memory storage module of the memory storage module(s), and/or the non-transitory memory storage module(s) storing the one or more databases or the contents of that particular database can be spread across multiple ones of the memory storage module(s) and/or non-transitory memory storage module(s) storing the one or more databases, depending on the size of the particular database and/or the storage capacity of the memory storage module(s) and/or non-transitory memory storage module(s). In various embodiments, databases can be stored in a cache (e.g., MegaCache) for immediate retrieval on-demand. The one or more databases can each comprise a structured (e.g., indexed) collection of data and can be managed by any suitable database management systems configured to define, create, query, organize, update, and manage database(s). Exemplary database management systems can include MySQL (Structured Query Language) Database, PostgreSQL Database, Microsoft SQL Server Database, Oracle Database, SAP (Systems, Applications, & Products) Database, IBM DB2 Database, and/or NoSQL Database.

Meanwhile, communication between web server 201, medical provider device 202, patient device 203, and/or the one or more databases can be implemented using any suitable manner of wired and/or wireless communication. Accordingly, system 200 can comprise any software and/or hardware components configured to implement the wired and/or wireless communication. Further, the wired and/or wireless communication can be implemented using any one or any combination of wired and/or wireless communication network topologies (e.g., ring, line, tree, bus, mesh, star, daisy chain, hybrid, etc.) and/or protocols (e.g., personal area network (PAN) protocol(s), local area network (LAN) protocol(s), wide area network (WAN) protocol(s), cellular network protocol(s), powerline network protocol(s), etc.). Exemplary PAN protocol(s) can comprise Bluetooth, Zigbee, Wireless Universal Serial Bus (USB), Z-Wave, etc.; exemplary LAN and/or WAN protocol(s) can comprise Institute of Electrical and Electronic Engineers (IEEE) 802.3 (also known as Ethernet), IEEE 802.11 (also known as Wi-Fi), etc.; and exemplary wireless cellular network protocol(s) can comprise Global System for Mobile Communications (GSM), General Packet Radio Service (GPRS), Code Division Multiple Access (CDMA), Evolution-Data Optimized (EV-DO), Enhanced Data Rates for GSM Evolution (EDGE), Universal Mobile Telecommunications System (UMTS), Digital Enhanced Cordless Telecommunications (DECT), Digital AMPS (IS-136/Time Division Multiple Access (TDMA)), Integrated Digital Enhanced Network (iDEN), Evolved High-Speed Packet Access (HSPA+), Long-Term Evolution (LTE), WiMAX, etc. The specific communication software and/or hardware implemented can depend on the network topologies and/or protocols implemented, and vice versa. In many embodiments, exemplary communication hardware can comprise wired communication hardware including, for example, one or more data buses, such as, for example, universal serial bus(es), one or more networking cables, such as, for example, coaxial cable(s), optical fiber cable(s), and/or twisted pair cable(s), any other suitable data cable, etc. Further exemplary communication hardware can comprise wireless communication hardware including, for example, one or more radio transceivers, one or more infrared transceivers, etc. Additional exemplary communication hardware can comprise one or more networking components (e.g., modulator-demodulator components, gateway components, etc.).

Turning ahead in the drawings, FIG. 3 illustrates a block diagram of a system 300 that can be employed for predicting vaccine sentiment, as described in greater detail below. System 300 is merely exemplary and embodiments of the system are not limited to the embodiments presented herein. System 300 can be employed in many different embodiments or examples not specifically depicted or described herein. In some embodiments, certain elements or modules of system 300 can perform various procedures, processes, and/or activities. In these or other embodiments, the procedures, processes, and/or activities can be performed by other suitable elements or modules of system 300.

Generally speaking, system 300 can be implemented with hardware and/or software. Part or all of the hardware and/or software implemented in system 300 can be conventional or part or all of the hardware and/or software can be customized (e.g., optimized) for implementing part or all of the functionality of system 300 described herein. When implemented as software, one or more elements of system 300 can be emulated (e.g., reproduced functionally and/or by action via software). For example, a virtual machine having one or more elements described below can be instantiated on one or more elements of system 200 (FIG. 2).

When implemented as hardware, one or more of the elements of system 300 can be coupled together using one or more chassis configured to hold one or more circuit boards and/or serial bus(es). These boards and buses allow the various elements of system 300 to communicate amongst each other to accomplish their intended purposes. While elements of system 300 are described below individually, each can also be integrated into one or more chassis, circuit boards, and/or buses of system 300. On the other hand, one or more elements of system 300 can also be removable (e.g., via a PCI slot on a motherboard and/or a USB port). One or more elements of system 300 may also be integrated and/or embedded in a different machine or manufacture. Although specific constructions of boards and buses within system 300 are not shown, it should be understood that their construction can be tied to a form factor selected for system 300.

System 300 can take a number of different form factors based on its implementation. For example, system 300 can be implemented as a desktop computer, a laptop computer, a mobile device, and/or a wearable device as described herein. Further, system 300 can comprise a single computer, a single server, a cluster or collection of computers or servers, or a cloud of computers or servers. Typically, a cluster or collection of servers can be used when the demand on 300 exceeds the reasonable capability of a single server or computer, when a distributed structure for system 300 is desired, and/or when parallel computing is desired.

In many embodiments, system 300 can comprise a processor 301, a memory storage 302, an input device 303, a graphics adapter 304, a display device 305, a graphical user interface (GUI) 306, a network adapter 307, a GPS 308, and/or an audio output 309.

Generally speaking, processor 301 can comprise any type of computational circuit. For example, processor 301 can comprise a microprocessor, a microcontroller, a controller, a complex instruction set computing (CISC) microprocessor, a reduced instruction set computing (RISC) microprocessor, a very long instruction word (VLIW) microprocessor, a graphics processor, a digital signal processor, application specific integrated circuits (ASICs), etc. Processor 301 can be configured to implement (e.g., run) computer instructions (e.g., program instructions) stored on memory devices in system 300. At least a portion of the program instructions, stored on these devices, can be suitable for carrying out at least part of the techniques and methods described herein. Architecture and/or design of processor 301 can be compliant with any of a variety of commercially distributed architecture families. Processor 301 can be configured to perform parallel computing in combination with other elements of system 300 and/or additional processors. Generally speaking, parallel computing can be seen as a technique where multiple elements of system 300 are used to perform calculations simultaneously. In this way, complex and repetitive tasks (e.g., training a predictive algorithm) can be performed faster and with less processing power than without parallel computing.

Generally speaking, memory storage 302 can comprise non-volatile memory (e.g., read only memory (ROM)) and/or volatile memory (e.g., random access memory (RAM)). The non-volatile memory can be removable and/or non-removable non-volatile memory. Meanwhile, RAM can comprise dynamic RAM (DRAM), static RAM (SRAM), or some other type of RAM. Further, ROM can include mask-programmed ROM, programmable ROM (PROM), one-time programmable ROM (OTP), erasable programmable read-only memory (EPROM), electrically erasable programmable ROM (EEPROM) (e.g., electrically alterable ROM (EAROM) and/or flash memory), or some other type of ROM. Memory storage 302 can comprise non-transitory memory and/or transitory memory. All or a portion of memory storage 302 can be referred to as memory storage module(s) and/or memory storage device(s). Memory storage 302 can have a number of form factors when used in system 300. For example, memory storage 302 can comprise a magnetic disk hard drive, a solid state hard drive, a removable USB storage drive, a RAM chip, etc.

Memory storage 302 can be encoded with a wide variety of computer code configured to operate system 300. For example, portions of memory storage 302 can be encoded with a boot code sequence suitable for restoring system 300 to a functional state after a system reset. As another example, portions of memory storage 302 can comprise microcode such as a Basic Input-Output System (BIOS) operable with elements of system 300. Further, portions of the memory storage 302 can comprise an operating system (e.g., a software program that manages the hardware and software resources of a computer and/or a computer network). The BIOS can be configured to initialize and test components of system 300 and load the operating system. Meanwhile, the operating system can perform basic tasks such as, for example, controlling and allocating memory, prioritizing the processing of instructions, controlling input and output devices, facilitating networking, and/or managing files. Exemplary operating systems can comprise software within the Microsoft® Windows®, Mac OS®, Apple® iOS®, Google® Android®, UNIX®, and/or Linux® series of operating systems.

Input device 303 can be configured to allow a user to interact and/or control elements of system 300. A number of devices and be used as input device 303 alone or in combination. For example, input device 303 can comprise a keyboard, a mouse, a touch screen, a microphone, a camera, etc. Input device 303 can be coupled to other elements of system 300 in a number of ways. For example, input device 303 can be coupled via a Universal Serial Bus (USB) port in a wired and/or wireless manner or via a specialized port (e.g., a PS/2 port) depending on the specific device. User inputs through input device 303 can come in a number of forms. For example, when input device 303 comprises a microphone, user input can be received via voice commands and/or a speech to text algorithm. As another example, when input device 303 comprises a camera, user input can be received via bodily movements that are captured and interpreted by system 300.

Generally speaking, graphics adapter 304 can be configured to receive and/or generate one or more elements for display on display device 305. Exemplary embodiments of graphics adapter 304 can comprise devices within the NVIDIA® GeForce® and/or the AMD® RX® series of video cards. In many embodiments, a chipset present on graphics adapter 304 can be configured to perform similar, simultaneous computations in a manner more efficient than other chipsets. For example, rendering a 3D scene on graphics adapter 304 can involve repeated geometric calculations performed in parallel to generate the 3D scene. As another example, repeated mathematical calculations involved in training a predictive algorithm can be performed in parallel on graphics adapter 304 more efficiently thank on processor 301. Display device 305 can receive and display signals from graphics adapter 304. A number of devices can be used as display device 305. For example, display device 305 can comprise a computer monitor, a television, a touch screen display, a heads up display (HUD) medium, etc.

In some embodiments, display device 304 can optionally display graphical user interface (GUI). A GUI can be a part of and/or displayed by one or more of web server 201 (FIG. 2), medical provider device 202 (FIG. 2), and/or patient device 203 (FIG. 2). With regards to form, GUI can comprise text and/or graphics (image) based user interfaces. For example, GUI can comprise a heads up display (HUD). When GUI comprises a HUD, GUI can be projected onto a medium (e.g., glass, plastic, metal, etc.), displayed in midair as a hologram, and/or displayed on display device 305. GUI can be color, black and white, and/or greyscale. GUI can be implemented as an application running on a computer system, such as computer system 300, web server 201 (FIG. 2), medical provider device 202 (FIG. 2), and/or patient device 203 (FIG. 2). GUI can also comprise a website accessed through a network (e.g., internet 220). For example, GUI can comprise vaccination website described herein. When GUI allows for modification and/or changes to one or more settings in system 300, it can be referred to as an administrative (e.g., back end) GUI. GUI can also be displayed as or on a virtual reality (VR) and/or augmented reality (AR) system or display. GUI can receive a number of interactions from a user via input device 303. For example, an interaction with a GUI can comprise a click, a look, a selection, a grab, a view, a purchase, a bid, a swipe, a pinch, a reverse pinch, etc.

Network adapter 307 can be configured to connect system 300 to a computer network by wired communication (e.g., a wired network adapter) and/or wireless communication (e.g., a wireless network adapter). Network adapter 307 can be integrated into one or more chassis, circuit boards, and/or buses or be removable (e.g., via a PCI slot on a motherboard). For example, network adapter 307 can be implemented via one or more dedicated communication chips configured to receive various protocols of wired and/or wireless communications.

GPS 308 can comprise a chipset and/or module configured to communicate with a satellite based location system configured to provide location and time information. (e.g., GPS 230). This location and time information can then be used to determine a location of system 300. Audio output 309 can be configured to receive and/or generate one or more audio signals for play through a speaker. Exemplary audio outputs 309 can comprise an audio card.

For simplicity and clarity of illustration, the drawing figures illustrate the general manner of construction, and descriptions and details of some features and techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denote the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

The terms “couple,” “coupled,” “couples,” “coupling,” and the like should be broadly understood and refer to connecting two or more elements mechanically and/or otherwise. Two or more electrical elements may be electrically coupled together, but not be mechanically or otherwise coupled together. Coupling may be for any length of time, e.g., permanent or semi-permanent or only for an instant. “Electrical coupling” and the like should be broadly understood and include electrical coupling of all types. The absence of the word “removably,” “removable,” and the like near the word “coupled,” and the like does not mean that the coupling, etc. in question is or is not removable.

As defined herein, two or more elements are “integral” if they are comprised of the same piece of material. As defined herein, two or more elements are “non-integral” if each is comprised of a different piece of material.

As defined herein, “real-time” can, in some embodiments, be defined with respect to operations carried out as soon as practically possible upon occurrence of a triggering event. A triggering event can include receipt of data necessary to execute a task or to otherwise process information. Because of delays inherent in transmission and/or in computing speeds, the term “real time” encompasses operations that occur in “near” real time or somewhat delayed from a triggering event. In a number of embodiments, “real time” can mean real time less a time delay for processing (e.g., determining) and/or transmitting data. The particular time delay can vary depending on the type and/or amount of the data, the processing speeds of the hardware, the transmission capability of the communication hardware, the transmission distance, etc. However, in many embodiments, the time delay can be less than approximately one second, two seconds, five seconds, or ten seconds.

As defined herein, “approximately” can, in some embodiments, mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

Although systems and methods for predicting vaccine sentiment have been described with reference to specific embodiments, it will be understood by those skilled in the art that various changes may be made without departing from the spirit or scope of the disclosure. Accordingly, the disclosure of embodiments is intended to be illustrative of the scope of the disclosure and is not intended to be limiting. It is intended that the scope of the disclosure shall be limited only to the extent required by the appended claims. For example, to one of ordinary skill in the art, it will be readily apparent that any element of FIGS. 1-3 may be modified, and that the foregoing discussion of certain of these embodiments does not necessarily represent a complete description of all possible embodiments. For example, one or more of the procedures, processes, or activities of FIG. 1 may include different procedures, processes, and/or activities and be performed by many different modules, in many different orders.

All elements claimed in any particular claim are essential to the embodiment claimed in that particular claim. Consequently, replacement of one or more claimed elements constitutes reconstruction and not repair. Additionally, benefits, other advantages, and solutions to problems have been described with regard to specific embodiments. The benefits, advantages, solutions to problems, and any element or elements that may cause any benefit, advantage, or solution to occur or become more pronounced, however, are not to be construed as critical, required, or essential features or elements of any or all of the claims, unless such benefits, advantages, solutions, or elements are stated in such claim.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

What is claimed is:

1. A system comprising:
one or more processors; and
one or more non-transitory computer-readable storage devices storing computing instructions configured to run on the one or more processors and cause the one or more processors to perform:
generating one or more training datasets;
training a predictive algorithm on the one or more training datasets;
receiving new patient data of a patient;
determining, using the trained predictive algorithm and the new patient data, a vaccine sentiment score;
applying a label to the patient using the vaccine sentiment score; and
coordinating displaying a vaccine communication using the label.

2. The system of claim 1, wherein the predictive algorithm comprises a machine learning algorithm.

3. The system of claim 1, wherein the predictive algorithm comprises a decision tree algorithm.

4. The system of claim 3, wherein the decision tree algorithm comprises a gradient boosted decision tree.

5. The system of claim 1, wherein the computing instructions are further configured to run on the one or more processors and perform:
receiving an updated training data set; and
re-training the predictive algorithm.

6. The system of claim 1, wherein:
the patient comprises a plurality of patients in a geographic area; and
the vaccine communication comprises a vaccine sentiment score for the patients in the geographic area.

7. The system of claim 1, wherein the computing instructions are further configured to run on the one or more processors and perform:
identifying, using the predictive algorithm and the new patient data, a vaccine sentiment score for a single vaccine or a series of multi-dose vaccines.

8. A method implemented via execution of computing instructions configured to run at one or more processors and configured to be stored at non-transitory computer-readable media, the method comprising:
generating one or more training datasets;
training a predictive algorithm on the one or more training datasets;
receiving new patient data of a patient;
determining, using the trained predictive algorithm and the new patient data, a vaccine sentiment score;
applying a label to the patient using the vaccine sentiment score; and
coordinating displaying a vaccine communication using the label.

9. The method of claim 8, wherein the predictive algorithm comprises a machine learning algorithm.

10. The method of claim 8, wherein the predictive algorithm comprises a decision tree algorithm.

11. The method of claim 10, wherein the decision tree algorithm comprises a gradient boosted decision tree.

12. The method of claim 8 further comprising:
receiving an updated training data set; and
re-training the predictive algorithm.

13. The method of claim 8, wherein:
the patient comprises a plurality of patients in a geographic area; and
the vaccine communication comprises a vaccine sentiment score for the patients in the geographic area.

14. The method of claim 8 further comprising:
identifying, using the predictive algorithm and the new patient data, a vaccine sentiment score for a single vaccine or a series of multi-dose vaccines.

15. An article of manufacture comprising a non-transitory, tangible computer readable storage medium having instructions stored thereon that, in response to execution by a computer based system, cause the computer based system to perform operations comprising:
generating one or more training datasets;
training a predictive algorithm on the one or more training datasets;
receiving new patient data of a patient;
determining, using the trained predictive algorithm and the new patient data, a vaccine sentiment score;
applying a label to the patient using the vaccine sentiment score; and
coordinating displaying a vaccine communication using the label.

16. The article of manufacture of claim 15, wherein the predictive algorithm comprises a machine learning algorithm.

17. The article of manufacture of claim 15, wherein the predictive algorithm comprises a decision tree algorithm.

18. The article of manufacture of claim 17, wherein the decision tree algorithm comprises a gradient boosted decision tree.

19. The article of manufacture of claim 15, wherein the operations further comprise:
receiving an updated training data set; and
re-training the predictive algorithm.

20. The article of manufacture of claim 15, wherein:
the patient comprises a plurality of patients in a geographic area; and
the vaccine communication comprises a vaccine sentiment score for the patients in the geographic area.
